(19) United States
(12) Patent Application Publication
(10) Pub. No.: US 2011/0027911 A1
(43) Pub. Date: Feb. 3, 2011

(54) SOLUBILIZATION AND STUDY OF MEMBRANE PROTEINS

(75) Inventors: A. Joshua Wand, Philadelphia, PA (US); Ronald W. Peterson, Media, PA (US); Joseph M. Kielec, Boston, MA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: THE TRUSTEES OF THE UNIVERSITY OF PENNSYLVANIA, Philadelphia, PA (US)

(21) Appl. No.: 12/812,152

(22) PCT Filed: Jan. 9, 2009
(86) PCT No.: PCT/US09/30575

§ 371 Date: Oct. 6, 2010

Related U.S. Application Data
(60) Provisional application No. 61/020,280, filed on Jan. 10, 2008.

(51) Int. Cl. G01N 33/53; G01J 3/00

(52) U.S. Cl. 436/501; 356/300

(57) ABSTRACT

Provided are methods for solubilizing a membrane protein in order to provide substantially homogeneous membrane proteins that are solubilized within reverse micelle systems at concentrations that are suitable for analytical study of such membrane proteins, including nuclear magnetic resonance spectroscopy. Also provided are substantially homogeneous membrane proteins that are solubilized within a reverse micelle system, as well as methods for the study of solubilized membrane proteins, and for the screening of drug candidates that target such membrane proteins.

### CROSS-REFERENCE TO RELATED APPLICATIONS

This application claims the benefit of priority to U.S. Provisional Patent Application Ser. No. 61/020,280, filed Jan. 10, 2008, which is herein incorporated by reference in its entirety.

### GOVERNMENT RIGHTS

Research leading to the disclosed invention was funded, in part, by the National Science Foundation, pre-doctoral fellowship grant number 2004016452 to J. M. Kielec, who is also an National Institutes of Health pre-doctoral trainee (GM 08275). Accordingly, the United States Government may have rights in the invention described herein.

### FIELD OF THE INVENTION

The present invention pertains to the solubilization and study of membrane proteins, including integral membrane proteins and anchored membrane proteins.

### BACKGROUND OF THE INVENTION

The detailed structural characterization of membrane proteins, including integral membrane proteins and anchored membrane proteins, has traditionally been fraught with problems. For example, only a small number of integral membrane proteins have been successfully characterized using solution nuclear magnetic resonance (NMR) methods. With respect to certain membrane proteins, a previous approach has involved the solubilization of recombinantly expressed protein in detergent micelles. These assemblies are quite large, even for relatively small proteins, often reaching 80 kDa and beyond. To undertake triple resonance NMR of such large and therefore slowly tumbling systems, the TROSY effect in combination with perdeuteration must be employed. In addition to the overall loss of potential structural information (derived from ¹H interactions), there is a somewhat ironic and very debilitating barrier presented by this approach: the back-exchange of amide sites with hydrogen following expression in 100% D₂O is generally problematic for membrane proteins. This is because they most often cannot be unfolded and refolded, which is the best and perhaps only way of fully back exchanging hydrogen for deuterium at amide sites. Without the amide NH, all is usually lost.

Previous efforts have included the solubilization of bovine rhodopsin, a membrane-embedded protein with cytoplasmic, membrane, and extracellular portions, in heavy organics using reverse micelle surfactants. Darszon, A., Strasser, R. J., and Montal, M. (1979) Rhodopsin—phospholipid complexes in a polar environments: photochemical characterization. Biochemistry 18, 5205-13. This was done at very low concentrations of protein. Essentially, protein embedded in natural membrane (i.e., with phospholipids) was injected into heavy organic solution of reverse micelle surfactant. See Ramakrishnan, V. R., Darszon, A., and Montal, M. (1983) A small angle x-ray scattering study of a rhodopsin-lipid complex in hexane. J Biol Chem 258, 4857-60. The structure of such a complex was further characterized by a study involving small angle x-ray scattering experiments on myelin in the anionic detergent AOT. Binks, B. P., Chatenay, D., Nicot, C., Urbach, W., and Waks, M. (1989) Structural parameters of the myelin transmembrane proteolipid in reverse micelles. Biophys J 55, 949-55. Low angle x-ray scattering profiles indicated the existence of a dumbbell-like structure similar. This approach has proven not to be viable in connection with the use of NMR-adequate concentrations of protein (i.e., protein concentrations at the millimolar scale). The difficulty was that at high protein concentrations the amount of either natural lipids or aqueous detergents present effectively ruined the reverse micelle surfactant phase diagram leading to aggregation. Although excellent spectra were obtained at very low concentrations, it was not possible to reach the concentrations necessary for high quality triple resonance spectroscopy.

Aside from proteins of a truly integral nature, many membrane proteins are anchored to cellular membranes via a carboxy-terminal helical segment. In addition, perhaps 5 to 10% of all cellular proteins bind to the membrane bilayer by insertion of a covalently attached lipid modification. Covalent attachment of fatty acids such as myristate and palmitate occurs on a wide variety of viral and cellular proteins. Myristate, a fourteen carbon saturated fatty acid, and palmitate, a sixteen carbon saturated fatty acid, commonly serve as key elements of membrane targeting and anchoring of proteins. Anchoring clearly disturbs the dynamical organization of the bilayer. Reversible membrane binding, controlled through triggered exposure of lipid anchors, is central to protein-protein interactions mediating signal transduction at the membrane. In addition, lipid anchors are also now thought to be critical to the exit of viruses from the eukaryotic cell. Indeed, the coupled interaction of fatty acids covalently attached to the HIV matrix protein with the membrane and phosphoinositides embedded in target membranes is thought to be central to its localization to the plasma membrane which in turn begins to the assembly of the immature virus. Accordingly, this initiation of virion assembly is argued to be a prime candidate for pharmaceutical intervention.

Structural knowledge of this process would be of obvious utility. However, detailed characterization of the structural features of the myristoylated matrix protein, both by itself and in complex with PIP2 has been frustrated by the poor solution properties of both the protein and PIP2. Saad, J. S., Miller, J., Tai, J., Kim, A., Ghanam, R. H., and Summers, M. F. (2006) Structural basis for targeting HIV-1 Gag proteins to the plasma membrane for virus assembly. Proc Natl Acad Sci USA 103, 11364-9. Often what is done is to simply remove the portion of the protein that carries the lipid anchor leaving a highly soluble and well-behaved protein. This, however, obviously precludes examination of the role of the anchor itself and the interaction of the anchored protein with molecules presented by the membrane. A review of the pertinent literature reveals that high resolution information about the nature of lipidated proteins is remarkably sparse perhaps because of this inherent difficulty.

There remains a great need for techniques that enable the full suite of nuclear magnetic resonance spectroscopy experiments and other analytical and screening techniques in connection with membrane proteins, which carry out many essential cell functions and by some estimates represent more than a quarter of all proteins encoded in genomes (see, e.g., Liu J F & Rost B (2001) Comparing function and structure between entire proteoms. Protein Sci. 10: 1970-1979). Traditional methods for the transfer of membrane proteins from the lipid bilayer to processing media fail to provide substantially homogenous samples and do not yield samples in which the concentration of solubilized protein approaches the level required for NMR study.

### SUMMARY OF THE INVENTION

Provided are methods for solubilizing a membrane protein comprising blending a first surfactant comprising an aqueous detergent with a sample of the membrane protein to form an aqueous detergent-protein mixture; concentrating the mixture; and, combining the concentrated mixture with an organic solvent and either a second surfactant or an additional quantity of the first surfactant to form a reverse micelle system in which the membrane protein is solubilized, and wherein the solubilized membrane protein is substantially homogeneous.

Also disclosed are methods for assessing a drug candidate comprising contacting the drug candidate with a membrane protein that is solubilized within a reverse micelle system, and performing at least one of assessing the binding between said drug candidate and said membrane protein, determining whether said drug candidate modulates the conformation of said membrane protein, determining whether said drug candidate modulates the degradation characteristics of said membrane protein, and determining whether said drug candidate modulates post-translational modification of said membrane protein.

There are also disclosed substantially homogeneous membrane proteins that are solubilized within a reverse micelle system comprising a first surfactant, the first surfactant comprising an aqueous detergent; an organic solvent; and, optionally, a second surfactant.

Also provided are methods for performing spectroscopic analysis of a membrane protein comprising placing a substantially homogeneous sample of the membrane protein in a spectroscopic analytical instrument, wherein the membrane protein is solubilized within a reverse micelle system comprising a first surfactant, the first surfactant comprising an aqueous detergent; an organic solvent; and a second surfactant; and, performing spectroscopic analysis of the solubilized membrane protein.

There are also disclosed methods for solubilizing an anchored membrane protein comprising combining the protein with a surfactant capable of forming reverse micelles. Also provided are anchored membrane proteins that are solubilized within a reverse micelle system.

Also disclosed herein are methods for performing spectroscopic analysis of an anchored membrane protein comprising placing a sample of the anchored membrane protein in a spectroscopic analytical instrument, wherein the anchored membrane protein is solubilized within a reverse micelle system; and, performing spectroscopic analysis of the anchored membrane protein.

There are also disclosed methods for assessing a drug candidate comprising contacting the drug candidate with an anchored membrane protein that is solubilized within a reverse micelle system; and performing at least one of assessing the binding between said drug candidate and said anchored membrane protein, determining whether said drug candidate modulates the conformation of said anchored membrane protein, determining whether said drug candidate modulates the degradation characteristics of said anchored membrane protein, and determining whether said drug candidate modulates post-translational modification of said anchored membrane protein.

### BRIEF DESCRIPTION OF THE DRAWINGS

FIG. 1 provides a schematic representation of the postulated organization of a reverse micelle particle solubilizing a membrane protein.

FIG. 2 illustrates the path of optimization of KcsA sample composition, in which ¹⁵N-HSQC spectra are used to monitor the progress of optimization.

FIG. 3 depicts the distribution of backbone ¹⁵N T₂ relaxation times in KcsA solubilized in CTAB/DHAB reverse micelles at 35° C.

FIGS. 4A and 4B provide the results of three dimensional NMR spectroscopy of reverse micelle solubilized KcsA; the sample was 0.2 mM in KcsA monomers and the data was collected at 35° C. on a 600 MHz spectrometer equipped with a cold probe (EB S/N 3300:1).

FIGS. 5A and 5B depict the ¹⁵N-HSQC spectra of the matrix domain (MA) of the Gag protein, without (myr-MA) the N-terminus myrisotyl group in water, and with (myr+MA) the N-terminus myrisotyl group and encapsulated within reverse micelles, respectively.

FIG. 6 illustrates the binding of PIP2 to myr+MA, including a superimposition of ¹⁵N-HSQC of the encapsulated myr+MA protein at various levels of added PIP2; local perturbation indicates specific binding to the protein.

FIG. 7A shows the ¹⁵N HSQC spectrum of an aqueous solution of myristoylated ¹⁵N recoverin in the Ca²⁺ free state for comparison. FIGS. 7B and 7C depict the ¹⁵N HSQC of CTAB reverse micelle encapsulation of myristoylated recoverin in the absence (B) and presence (C) of Ca²⁺. The residues G115 and G79 are indicated as key residues involved in the sequestration of the myristoyl tail of recoverin.

### DETAILED DESCRIPTION OF ILLUSTRATIVE EMBODIMENTS

The present invention pertains to novel methods for the solubilization of membrane proteins, solubilized membrane proteins, and methods for the study of solubilized membrane proteins. The instant methods and products employ new reverse micelle systems that provide solubilized, substantially homogeneous membrane proteins, including integral membrane proteins and anchored membrane proteins. The solubilization of membrane proteins in accordance with the instant invention enables the use of spectroscopic analysis, including nuclear magnetic resonance (NMR) spectroscopy, in association therewith, and permits high-throughput screening of drug candidates, among other applications that rely on the successful transfer of a membrane protein from its natural lipidated environment to a controlled, in vitro environment while still maintaining the native conformation of the protein.

U.S. Pat. No. 6,198,281 introduced the basic idea of improving the NMR performance of large proteins by actively increasing the rate of molecular tumbling. That approach took advantage of the Stokes-Einstein relationship between solvent viscosity and diffusional reorientation of a sphere, and involved the solvation of a reverse micelle-forming surfactant in a low viscosity solvent, followed by the transfer or distribution of a hydrated protein, such as cytochrome c, into the surfactant-solvent phase via encapsulation. However, that approach did not achieve reverse micelle systems in which the membrane proteins are substantially homogeneous, or reverse micelle systems in which membrane proteins were present in the concentration required for NMR applications.

The present invention is complementary to and somewhat competitive with purely spectroscopy (TROSY) or chemical (deuteration) approaches. It is unique in its ability to study proteins of marginal stability through a confined space effect (see Peterson et al. (2004) JAGS 126, 9498). A nonlimiting example of the advantages of the present approach is that the full suite of NMR experiments can be employed and fully exploited. This is not true for TROSY and/or deuteration approaches. The specific disclosure made herein pertains to methods of encapsulation (or more properly, solubilization) of membrane proteins, including integral and anchored membrane proteins, and provides distinct advantages over aqueous detergent solubilization of membrane proteins.

Provided are methods for solubilizing a membrane protein comprising blending a first surfactant comprising an aqueous detergent with a sample of the membrane protein to form an aqueous detergent-protein mixture; concentrating the mixture; and, combining the concentrated mixture with an organic solvent and either a second surfactant or an additional quantity of the first surfactant to form a reverse micelle system in which the membrane protein is solubilized, and wherein the solubilized membrane protein is substantially homogeneous.

As used herein, the term “membrane protein” refers to a membrane-associated protein of which at least one portion is embedded within the phospholipid cell membrane in the protein's natural state. For example, integral membrane proteins may be nearly fully contained within the cell membrane, or may have extracellular portions, cytoplasmic portions, or both. Anchored membrane proteins are characterized as having at least one hydrophobic “anchor” portion that is embedded within the cell membrane, and either a cytoplasmic or extracellular portion. An example of an anchored membrane protein is the HIV1-matrix protein (a domain of the so-called Gag protein). Other membrane protein types, and specific examples thereof, are readily appreciated by those skilled in the art. In accordance with the instant invention, the membrane protein is faithfully extracted or transferred from the membrane bilayer into the aqueous detergent, which permits the membrane protein to retain its native conformation during the process of solubilization within the instant reverse micelle systems. Thus, membrane proteins for use in connection with the instant methods are preferably not isolated from the membrane bilayer prior to the solubilization process.

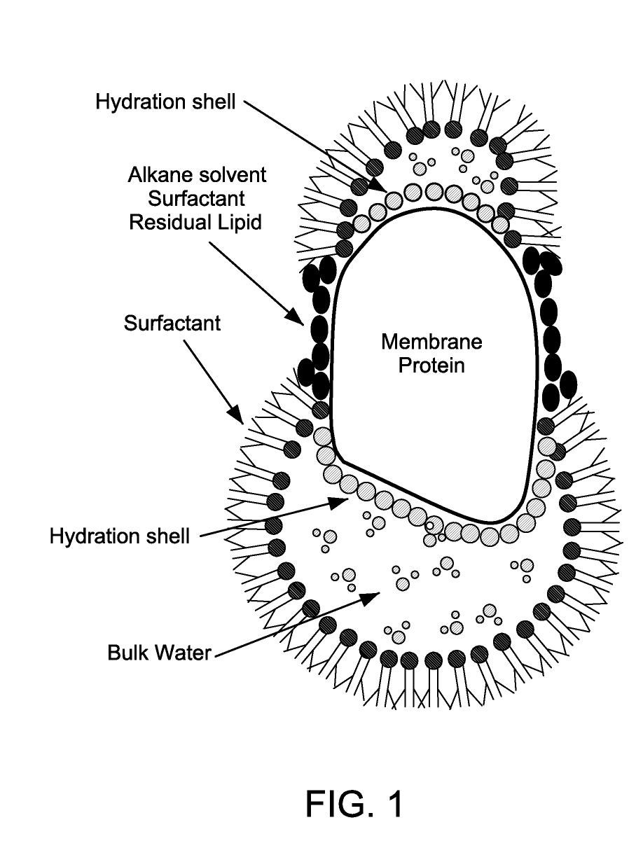

While not intending to be bound by any particular theory of operation, it is believed that a reverse micelle particle containing an membrane protein is quite different than that of an encapsulated soluble protein. There is some evidence that a reverse micelle particle containing an membrane protein corresponds to the structure shown in FIG. 1. It is believed that the distinct advantage of the reverse micelle system, in contrast to simple dissolution in organic solvents like chloroform, is that the system provides both hydrophobic (alkane solvent, surfactant tail; tightly bound lipid) and polar (surfactant head groups, water) molecules to support the structure of the protein. In FIG. 1, the schematic representation of the postulated organization of a reverse micelle particle solubilizing an integral membrane protein is based on low angle scattering data of M. Montal and coworkers. See, e.g., Darszon, A., Strasser, R. J., and Montal, M. (1979) Rhodopsin—phospholipid complexes in a polar environments: photochemical characterization. Biochemistry 18, 5205-13. FIG. 1 depicts a model in which hydration water is held in two partial reverse micelles spanning each polar face of the protein, and in which the hydrophobic region of the protein may be supported by surfactant, alkane solvent, and any tightly bound lipids that are carried through the purification.

When used to describe the solubilized membrane proteins of the present invention, the term “substantially homogenous” means that there is at least 90% homogeneity with respect to the conformation of the solubilized protein within the instant reverse micelle system. For example, when compared to the measured ¹⁵N-HSQC spectrum of the properly folded membrane protein in water, the ¹⁵N-HSQC spectrum of the protein in the reverse micelle system shows at least 90% conformity with the control spectrum. More particularly, if the membrane protein has 100 amino acid residues other than proline, 90 of the crosspeaks in the ¹⁵N-HSQC spectrum of the protein in the reverse micelle system are single, sharp peaks, rather than multiple peaks. Homogeneity therefore refers to the fidelity of the conformation of the solubilized membrane protein to the native conformation of the protein. Samples of solubilized membrane proteins can be inhomogeneous when the technique for transferring the membrane proteins from the lipid bilayer to the solubilization environment results in the disruption of the native folding of the protein; the instant invention, however, in providing substantially homogeneous solubilized membrane proteins, results from a “successful” transfer from the lipid bilayer to the instant reverse micelle systems. Those skilled in the art will readily appreciate other ways to assess the homogeneity of the solubilized membrane protein sample, such as by measuring the breadth of the cross-peaks in the ¹⁵N-HSQC spectrum, or other methods. One general approach is that “native like” spectra, i.e., sharp resonance lines without, for example, significant population of minor conformers, correspond to the native structure. Non-native states may be characterized by line broadening, multiple conformations, and/or other spectral features consistent with the presence of multiple states. This approach for analyzing the homogeneity of a membrane protein sample that is solubilized in a reverse micelle system is similar to that used in prior studies with respect to optimization of aqueous detergent solubilization of membrane proteins. See, e.g., Sanders, C. R. and Oxenoid, K. (2000) Customizing model membranes and samples for NMR spectroscopic studies of complex membrane proteins. Biochim Biophys Acta 1508, 129-145; Krueger-Koplin, R. D., et al. (2004) An evaluation of detergents for NMR structural studies of membrane proteins. Journal of Biomolecular Nmr 28, 43-57. In other embodiments of the present invention, the solubilized membrane proteins have at least 95% homogeneity in the reverse micelle systems. In still other embodiments, the solubilized membrane proteins have at least 98% homogeneity in the present reverse micelle systems.

In addition to the homogeneity of the membrane proteins that are solubilized within the instant reverse micelle systems, the present invention also provides reverse micelle systems in which the solubilized membrane protein is present in concentrations that are suitable for use with NMR spectroscopic analysis. Previous methods do not provide the protein concentrations necessary for high quality triple resonance spectroscopy, and in contrast the instant invention provide reverse micelle systems in which a membrane protein is present in an amount of at least about 0.1 mM. In other embodiments, the membrane protein can be present in the reverse micelle system in an amount of at least about 0.5 mM or at least about 1.0 mM. The present invention also provides reverse micelle systems in which the solubilized membrane protein is present in concentrations that are lower than those required for NMR spectroscopy, but are nonetheless suitable for use in connection with other types of structural analysis, such as other types of spectroscopic analysis. For example, fluorescence spectroscopy is much more sensitive than NMR spectroscopy, and can be performed using much lower concentrations of protein in a sample. The present invention is capable of providing protein concentrations in the instant reverse micelle systems that are in the range of 1 μM, 0.5 μM, 0.1 μM, or even as low as in the range of 1 nM. One skilled in the art may select the protein concentration that is optimal for the desired analytical method.

It has presently been discovered that the first surfactant for use in the instant methods should be of a “dual” nature: it should be capable of acting as an aqueous detergent, and should be able to function as a reverse micelle surfactant, i.e., should be capable of forming reverse micelles. Exemplary surfactants that are suitable in these respects include lauryldimethylamine oxide (LDAO), sodium bis(2-ethylhexyl) sulfosuccinate (AOT), N,N-dimethyl-N,N-dihexadecyl ammonium bromide (DRAB), and cetyltrimethylammoniumbromide (CTAB). Other surfactants that meet the dual requirements described above may be readily identified by those skilled in the art or may be determined by routine experimentation.

The blending of the first surfactant with the membrane protein may be performed in accordance with numerous variations that will be apparent to those skilled in the art. For example, the surfactant may be placed into a blending vessel in which the membrane protein had previously been placed, followed by mixing of the surfactant and protein contents. Mixing may be achieved using conventional methods, such as by manual or mechanical mild agitation or by using a magnetic stirring apparatus.

Once blending has occurred, the detergent-protein mixture is concentrated. The mixture may be concentrated to dryness. Substantially complete drying or partial drying can be achieved by any conventional method, such as by ambient evaporation, heat-induced evaporation, lyophilization, shaking or “washing” with saturated aqueous sodium chloride, drying agents (often inorganic salts), and the like. Other techniques for drying the detergent-protein mixture will be readily appreciated by those skilled in the art. Concentration of the detergent-protein mixture is performed because the solubility of micelles in water is less than that in organic solutions. The degree of concentration of the detergent-protein mixture may be dictated by the desired solubilized protein concentration in the final reverse micelle system. A simple calculation may be used to determine the extent to which the detergent-protein mixture should be concentrated:

$$\frac{Organic Volume + Aqueous Volume}{Aqueous Volume} = \frac{Final Protein Concentration}{Target Concentration}$$

wherein “organic volume” refers to the volume of the organic solvent added to the detergent-protein mixture, “aqueous volume” refers to the volume of the detergent-protein mixture to combine with the organic solvent to form the reverse micelle, “final protein concentration” refers to the final concentration of solubilized membrane protein in the reverse micelle system, and “target concentration” refers to the concentration of membrane protein in the detergent-protein mixture following the concentration step.

Following the concentration step, the concentrated mixture is combined with an organic solvent and either a second surfactant or an additional quantity of said first surfactant. The concentrated mixture may first be combined with the organic solvent, and then that combination may be combined with the second surfactant or additional quantity of said first surfactant, or the concentrated mixture may be combined with a mixture of the organic solvent and second surfactant or an additional quantity of said first surfactant. The act of combining may be performed by mixing the concentrated mixture with the other ingredients, either by adding the concentrated mixture to the organic solvent or organic solvent with surfactant, or vice versa. Mixing may be achieved through stirring, mild agitation, or other methods that will be readily appreciated by those skilled in the art. The organic solvent may be a straight-chain or branched alkane, examples being straight-chain ethane, propane, butane, pentane, hexane, septane, octane, nonane, and decane and their branched counterparts. Suitable alkanes include alkanes having fewer than 10 total carbon atoms. In other embodiments, the organic solvent may be an alkane having greater than 10 total carbon atoms. The alkane is preferably a low viscosity alkane. For purposes of the present application, “low viscosity” means that the compound has a viscosity less than about 300 μPa·s. For example, ethane has a viscosity of 35 μPa·s at 300° K and at a pressure of 4.7 MPa. Other suitable low viscosity materials are disclosed in U.S. Pat. No. 6,486,672, which is incorporated herein by reference in its entirety. Relatively few solvents of sufficiently low viscosity exist, and many of these require application of significant pressure to remain liquid at room temperature. Thus, samples using such solvents must be prepared and maintained under pressure during measurement. Significant pressure is required, often up to 8,000 psi. Although this could present a considerable challenge, self-sealing, high-quality NMR tubes that can withstand pressures up to 10,000 psi have been developed and are disclosed in U.S. Pat. No. 5,977,772, which is incorporated herein by reference in its entirety.

In some instances, the first surfactant is not fully effective by itself to provide a reverse micelle system in which a membrane protein is solubilized and achieves substantial homogeneity. In such instances, a second or co-surfactant should be used in accordance with the instant methods. For example, it has been discovered that while the surfactant CTAB possesses the dual characteristics of being an aqueous detergent and being able to function as a reverse micelle surfactant, it should be used with a second surfactant in order to create a reverse micelle system in which a substantially homogeneous sample of the membrane protein is solubilized. The second surfactant may be an aqueous detergent or an alcohol. For example, lauryldimethylamine oxide (LDAO), sodium bis(2-ethylhexyl) sulfosuccinate (AOT), cetyltrimethylammoniumbromide (CTAB), or N,N-dimethyl-N,N-dihexadecyl ammonium bromide (DHAB) are aqueous detergents that may serve as a second surfactant. Exemplary alcohols for use as the second surfactant include hexanol and pentanol. Suitable alcohols include straight-chain or branched alcohols having fewer than 10 total carbon atoms. In other embodiments, the alcohol may have greater than 10 total carbon atoms. The first surfactant and the second surfactant may be used in relative quantities that are roughly equal; for example, in one embodiment, the first surfactant may comprise cetyltrimethylammoniumbromide, and the second surfactant may comprise N,N-dimethyl-N,N-dihexadecyl ammonium bromide, and the first surfactant and said second surfactant may be present in the reverse micelle system in about a 1:1 ratio. In other embodiments, there may be an excess of first surfactant relative to the second surfactant, or vice versa. For example, the ratio of first surfactant to second surfactant in the instant reverse micelle systems may be about 1:1.25, 1:1.5, 1:2, 1:2.5, 1:3, 1:3.5, or 1:4, or may be about 1.25:1, 1.5:1, 2:1, 2.5:1, 3:1, 3.5:1, or 4:1.

Also disclosed are methods for assessing a drug candidate comprising contacting the drug candidate with a membrane protein that is solubilized within a reverse micelle system, and performing at least one of assessing the binding between said drug candidate and said membrane protein, determining whether said drug candidate modulates the conformation of said membrane protein, determining whether said drug candidate modulates the degradation characteristics of said membrane protein, and determining whether said drug candidate modulates post-translational modification of said membrane protein.

In accordance with the instant methods for assessing a drug candidate, the solubilization of the membrane protein within a reverse micelle system should be performed in accordance with the disclosed methods for solubilizing a membrane protein. For example, as described above, the solubilization of the membrane protein may be achieved by blending a first surfactant comprising an aqueous detergent with a sample of the membrane protein to form a detergent-protein mixture; concentrating the mixture; and combining said concentrated mixture with an organic solvent and either a second surfactant or an additional quantity of said first surfactant to form a reverse micelle system in which said membrane protein is solubilized, and wherein said solubilized membrane protein is substantially homogeneous. As described herein, methods for solubilizing anchored membrane proteins may include combining the anchored membrane protein with a surfactant that is capable of forming reverse micelles.

Those skilled in the art will recognize that numerous techniques may be used for performing at least one of assessing the binding between said drug candidate and said membrane protein, determining whether said drug candidate modulates the conformation of said membrane protein, determining whether said drug candidate modulates the degradation characteristics of said membrane protein, and determining whether said drug candidate modulates post-translational modification of said membrane protein. For example, numerous types of analytical spectroscopy can be used to assess the binding between molecules (e.g., determining the binding affinity), determining whether the conformation of a protein has been modulated, whether a protein has degraded or resists degradation, or determine if a protein has or has not undergone a post-translational modification. Other assays and techniques that are known among those skilled in the art may be used to perform a desired analysis step following the contacting of the drug candidate with the solubilized membrane protein. For example, numerous binding assays are well known among those skilled and the art and can be used to assess the binding between the drug candidate and the solubilized membrane protein. Likewise, protein degradation assays of various types are widely used among skilled artisans.

Also provided are substantially homogeneous membrane proteins that are solubilized within a reverse micelle system comprising a first surfactant, the first surfactant comprising an aqueous detergent; an organic solvent; and, optionally, a second surfactant. The substantially homogeneous membrane proteins may be prepared using the techniques and materials in accordance with the methods disclosed herein. In contrast with the present invention, previous methods have failed to provide substantially homogeneous, solubilized membrane proteins, and furthermore have failed to do in a manner that yields membrane proteins that are present in the reverse micelle system in concentrations that are suitable for NMR spectroscopic analysis, e.g., in an amount of at least about 0.1 mM. In some embodiments of the present substantially homogeneous membrane proteins, the membrane protein is present in the reverse micelle system in an amount of at least about 0.1 mM. In other embodiments, the substantially homogeneous membrane protein can be present in the reverse micelle system in an amount of at least about 0.5 mM or at least about 1.0 mM. The present invention also provides reverse micelle systems in which the solubilized membrane protein is present in concentrations that are lower than those required for NMR spectroscopy, but are nonetheless suitable for use in connection with other types of structural analysis, such as other types of spectroscopic analysis, for example, as described previously. The characteristics of the instant solubilized membrane proteins render them superior for purposes of spectroscopic analysis and drug screening, and therefore for a wide array of high-quality protein studies that have heretofore been impossible or subject to unacceptable limitations.

The present invention also provides methods for solubilizing an anchored membrane protein comprising combining the anchored membrane protein with a surfactant capable of forming reverse micelles, and optionally, an alcohol. Also disclosed are anchored membrane proteins that are solubilized within a reverse micelle system. The present solubilized anchored membrane proteins are preferably substantially homogeneous. The solubilization of anchored membrane proteins within reverse micelle systems have never before been accomplished, and as a result, many forms of study of solubilized anchored membrane proteins have not been possible. In anchored membrane proteins, the lipid anchor portion of the protein affects the conformation of the hydrophilic portion of the protein: when the anchor is removed from the rest of the protein, the hydrophilic portion of the protein adopts the same conformation as when the anchor is embedded in the lipid bilayer, and when an attempt is made to solubilize the protein in accordance with traditional methods, the anchor is improperly lipidated and folds within the hydrophilic portion of the protein, thereby causing the hydrophilic portion to adopt a conformation that is different from its native state. In contrast, the instant methods for solubilizing an anchored membrane protein permit the anchor portion to become embedded within the reverse micelle surfactant shell, and as a result there is no distortion of the hydrophilic portion of the protein because the anchor projects into the wall of the reverse micelle, rather than burying itself within the hydrophilic portion of the protein. Thus, the present solubilized anchored membrane proteins may be used in connection with any analytical technique that requires the retention of native protein folding, such as spectroscopic analytical techniques (e.g., fluorescent spectroscopy, NMR spectroscopy), or drug screening techniques. The solubilized anchored membrane proteins are also preferably present in the reverse micelle system in a sufficient concentration for NMR analysis, e.g., at or above 0.1 mM. The anchored membrane protein may be a recombinantly expressed protein. The solubilized anchored membrane protein may also be present in concentrations that are lower than those required for NMR spectroscopy, but are nonetheless suitable for use in connection with other types of structural analysis, such as other types of spectroscopic analysis. The reverse micelle system may comprise any surfactant that is capable of forming reverse micelles. Preferably, the surfactant is an aqueous detergent. The surfactant may be at least one of lauryldimethylamine oxide, sodium bis(2-ethylhexyl) sulfosuccinate, cetyltrimethylammoniumbromide, and N,N-dimethyl-N,N-dihexadecyl ammonium bromide.

In view of such advantageous characteristics of the instant solubilized membrane proteins (e.g., substantial homogeneity, solubilization at millimolar concentrations), there are also provided methods for performing spectroscopic analysis of a membrane protein comprising placing a substantially homogeneous sample of the membrane protein in a spectroscopic analytical instrument, wherein the membrane protein is solubilized within a reverse micelle system comprising a first surfactant, the first surfactant comprising an aqueous detergent; an organic solvent; and, optionally, a second surfactant; and, spectroscopic analysis of the solubilized membrane protein. As discussed previously, the membrane protein may be any variety of membrane-associated protein of which at least one portion is contained within the phospholipid cell membrane in the protein's natural state. Nonlimiting examples include integral membrane proteins and anchored membrane proteins. The substantially homogeneous, solubilized membrane protein of the instant method may be prepared in accordance with the methods disclosed herein. In other embodiments, there are provided methods for performing spectroscopic analysis of an anchored membrane protein comprising placing a sample of the anchored membrane protein in a spectroscopic analytical instrument, wherein the anchored membrane protein is solubilized within a reverse micelle system; and, performing spectroscopic analysis of the anchored membrane protein. The solubilization of the anchored membrane protein may be performed in accordance with the methods disclosed above. The sample of the anchored membrane protein is preferably substantially homogeneous.

In accordance with the instant methods, any suitable type of spectroscopic analysis may be used; nonlimiting examples include nuclear magnetic resonance, fluorescent spectroscopy, UV/VIS, small-angle X-ray scattering (SAXS), and the like. A number of various types of spectroscopic analytical instruments are known among those skilled in the art, and any such device may be used in accordance with the present methods. The technique for placing the membrane protein sample in the analytical instrument may vary according to the type of instrument to be used, and the instant methods are intended embrace any such technique. Likewise, the act of performing spectroscopic analysis will be carried out as appropriate with respect to the type of spectroscopic device or application, as will be readily appreciated by the skilled artisan.

The present invention is further defined in the following examples. It should be understood that these examples, while indicating embodiments of the invention, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and conditions.

### Example 1

### Solubilization of the KcsA Integral Membrane Protein

A two-part screening procedure was utilized to determine which water-soluble surfactants (LDAO, CTAB, AOT) gave reasonably dispersed (for a helical protein) ¹⁵N-HSQC spectra in water with an appropriate number of amide crosspeaks. Detergent-protein samples showing the best watermicelle spectra were then dried down and prepared for reverse micelles. A successive screening procedure took place in which the membrane protein reverse micelle preparations were studied for crosspeak number, dispersion, and signal-noise. The membrane protein for use in the present study was the KcsA potassium channel, a 52 kDa homotetramer. The MacKinnon construct was used for the transmembrane domain. This involves a C-terminal His-tag fusion that produces, upon cleavage of the tag and the C-terminal soluble domain with chymotrypsin, a protein of 125 amino acids that forms the integral membrane homotetramer of 52 kDa. The study made use of 20 mg per litre of minimal media, making relatively brute force optimization economically feasible. In the case of the KcsA potassium channel, the most-promising surfactant proved to be CTAB. This sole use of detergents compatible with reverse micelles heightened the encapsulation efficiency, improving the yield over previous efforts and making protein concentrations of up to 250 μM feasible.

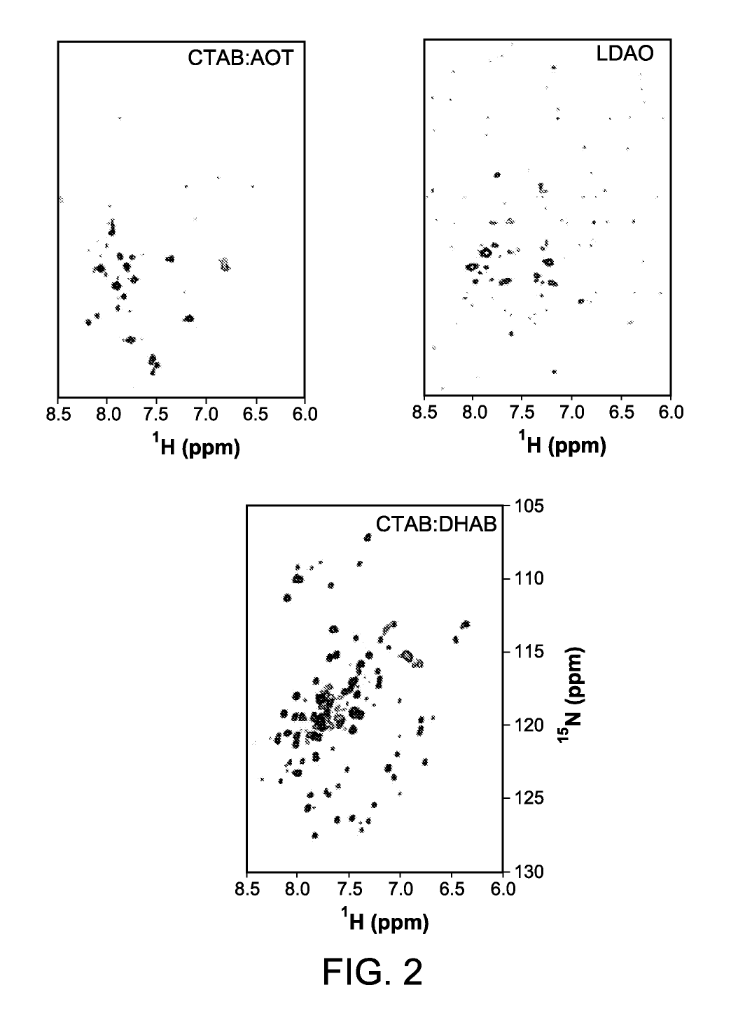

Additional optimization of the CTAB system included a survey of water-loading ratios, overall detergent levels for balance of T₂ times and signal-noise, co-surfactants, co-solvents, and preparation procedures to yield consistent, reproducible spectra. This optimization process revealed that the double-tailed surfactant DHAB, mixed in a 1:1 ratio to CTAB, heightened the quality of KcsA spectra, perhaps stabilizing the homotetrameric structure's transmembrane domains by contributing to the lateral pressure with its cylindrical structure. It was believed that this represented an optimal sample. An example of the KcsA reverse micelle survey is shown in FIG. 2, which depicts the use of ¹⁵N-HSQC spectra to monitor the progress of optimization. The thermodynamic hypothesis for protein folding stipulates that the native structure is unique and of lowest free energy. The nature of the energy landscape of proteins is such that perturbations away from the native structure necessarily compress the free energy gap between partially unfolded forms. This leads to broadening effects in the NMR spectrum due to interconversion between states.

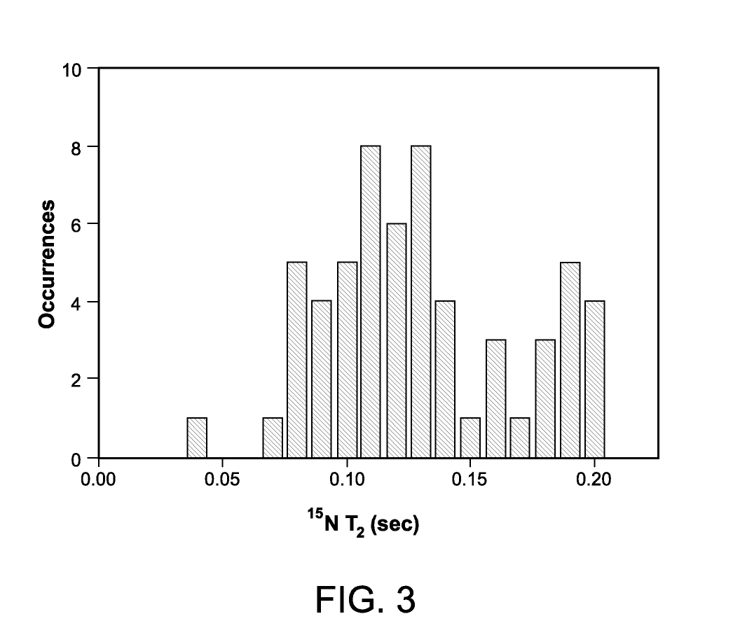

The sparse nature of the CTAB:AOT and LDAO preparations of KcsA were interpreted as being due to an inhomogeneous population of (partially) denatured proteins that are not interconverting at a rate greater than the chemical shift time scale. This is predicted by the thermodynamic hypothesis for protein folding and stability. The nature of the energy landscape of proteins is such that perturbations away from the native structure necessarily compress the free energy gap between partially unfolded forms. This has been observed many times in the study of protein stability using the so-called native state hydrogen exchange method. See, e.g., Fuentes, E. J. and Wand, A. J. (1998) Local stability and dynamics of apocytochrome b562 examined by the dependence of hydrogen exchange on hydrostatic pressure. Biochemistry 37, 9877-83; Fuentes, E. J. and Wand, A. J. (1998) Local dynamics and stability of apocytochrome b562 examined by hydrogen exchange. Biochemistry 37, 3687-98. This leads to broadening effects in the NMR spectrum due to interconversion between states. In contrast, the ¹⁵N-HSQC spectrum of KcsA in CTAB:DHAB (FIG. 2) is characteristic of a folded, homogenous and highly helical protein. This spectrum was obtained in liquid pentane and the high quality of the data suggests a rapidly tumbling molecule. Measurement of the backbone ¹⁵N-relaxation confirms this. While the average T₂ of ¹⁵N amide sites in KcsA solubilized in aqueous detergent micelles is on the order of 20 ms, those in the reverse micelle system are considerably longer. Indeed, they approach those of a 15-20 kDa protein (FIG. 3). This is an important result as it suggests that the standard triple resonance experiments can be applied without the limitations imposed by extensive deuteration or the TROSY effect. The spectrum of KcsA in the instant reverse micelle surfactant system is indicative of a folded and highly helical structure.

### Example 2

### Three Dimensional NMR Spectroscopy of Reverse Micelle Solubilized KcsA

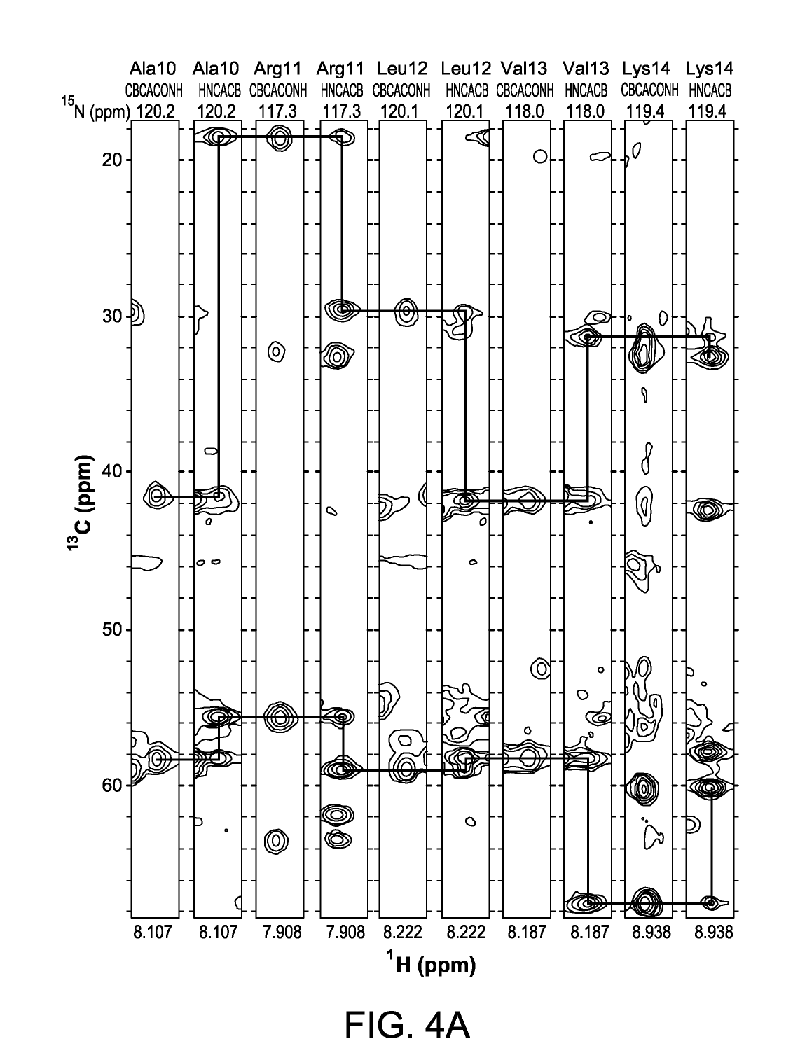

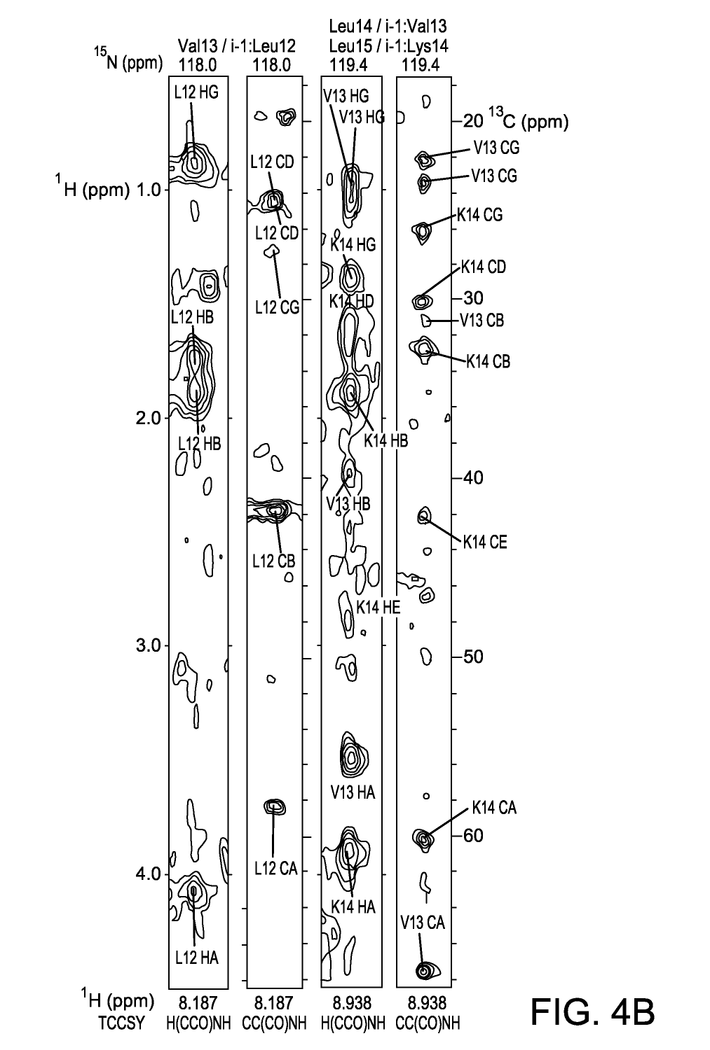

Shown in FIGS. 4A and 4B are strips from a HNCACB/CBCA(CO)NH pair outlining the assignment of a five residue section. These spectra were collected on a 0.2 mM (in monomers) KcsA sample in pentane/CTAB/DHAB at 35° C. The data was obtained at 600 MHz with a cold probe (EB S/N 3300:1). Each experiment required 3.5 days of instrument time.

FIG. 4A shows a short backbone walk of resonance assignments based on the CBCA(CO)NH/HNCACB pair of triple resonance spectra. Note the extensive degeneracy of the spectrum of this largely helical protein. FIG. 4B shows strips from three dimensional Hand C-evolved carbon TOCSY spectra resolved on the neighboring amide NH. These experiments are extremely sensitive to T₂ effects due to the small J-coupling employed to transfer coherence to the amide NH. Note the extensive transfer along long side chain spin systems.

These figures illustrate several points. On the one hand, the data is of good quality, certainly sufficient to reliably provide inter-residue correlations. On the other hand, the anticipated chemical shift degeneracy is indeed apparent. This will require use of complementary triple resonance data, for instance the NHCO/HN(CA)CO pair (see Sattler, M, Schleucher, J., and Griesinger, C. (1999) Heteronuclear multidimensional NMR experiments for the structure determination of proteins in solution employing pulsed field gradients. Progress in Nuclear Magnetic Resonance Spectroscopy 34, 93-158). FIGS. 4A and 4B also depict side chain correlations for several corresponding residues. Shown is ¹H and ¹³C resolved carbon-carbon TOCSY chains resolved on ¹⁵N-¹H of the adjacent residue. These experiments are very sensitive to T₂ and the spectra serve to illustrate that the samples are truly in the small protein tumbling regime. In short, everything so far obtained with this sample suggests that the native tertiary structure is present.

### Example 3

### Reverse Micelle Solubilization of the Gag Anchored Membrane Protein

In HIV, the so-called Gag protein is synthesized as a 55 kDa precursor that consists of four domains. One of these, the so-called matrix domain (MA) is myristoylated at the N-terminus and acts as a switch that helps regulate the binding of the Gag protein to the plasma membrane. Using solution NMR methods, others have solved the structures of the un-myristoylated (myr-MA) and myristoylated (myr+MA) forms of the isolated MA domain, hereafter termed the matrix protein. See Saad, J. S., et al. (2006) Structural basis for targeting HIV-1 Gag proteins to the plasma membrane for virus assembly. Proc Natl Acad Sci USA 103, 11364-9; Saad, J. S., et al. (2007) Mutations that mimic phosphorylation of the HIV-1 matrix protein do not perturb the myristyl switch. Prot Sci 16, 1793-7; Saad, J. S., et al. (2007) Point mutations in the HIV-1 matrix protein turn off the myristyl switch. J Mol Biol 366, 574-85. The isolated (monomeric) state of the myr+MA has a largely buried (sequestered) myristyl group whereas an oligomeric state (likely a trimer) has a (solvent) exposed myristyl group. Identification (targeting) of the plasma membrane has been thought to rely on the binding of PIP2 to the myr+MA.

Unfortunately, the poor solution properties of PI(4,5)P2 combined with oligomeric behavior of the MA protein precluded study of the natural “full length” PIP2. di-C8-PI(4,5)P2, a soluble truncated form of PIP2 was studied instead in experiments with the myristoylated exposed form of the protein. A second structure was obtained with di-C8-PI(4,5)P2 bound to the nonmyristoylated form of the protein. Since the topology of the protein changes very little, these structures were merged to obtain a model of the protein in its bound myristate exposed state. Though a very useful insight, it would be highly desirable to view the structural transitions of the binding of the myr+MA protein while bound to a membrane mimic.

The present study represented an attempt to determine if the inventive reverse micelle encapsulation strategy could prove useful in this context. A somewhat related study involved the use of non-specific modification with cholesterol to enhance the encapsulation efficiency of a soluble protein (Alberti, E., et al. (2002) Study of wheat high molecular weight 1Dx5 subunit by C-13 and H-1 solid-state NMR. II. Roles of nonrepetitive terminal domains and length of repetitive domain. Biopolymers 65, 158-168); a notable distinction is that that study did not involve a protein that is at least partially lipidated in its native state.

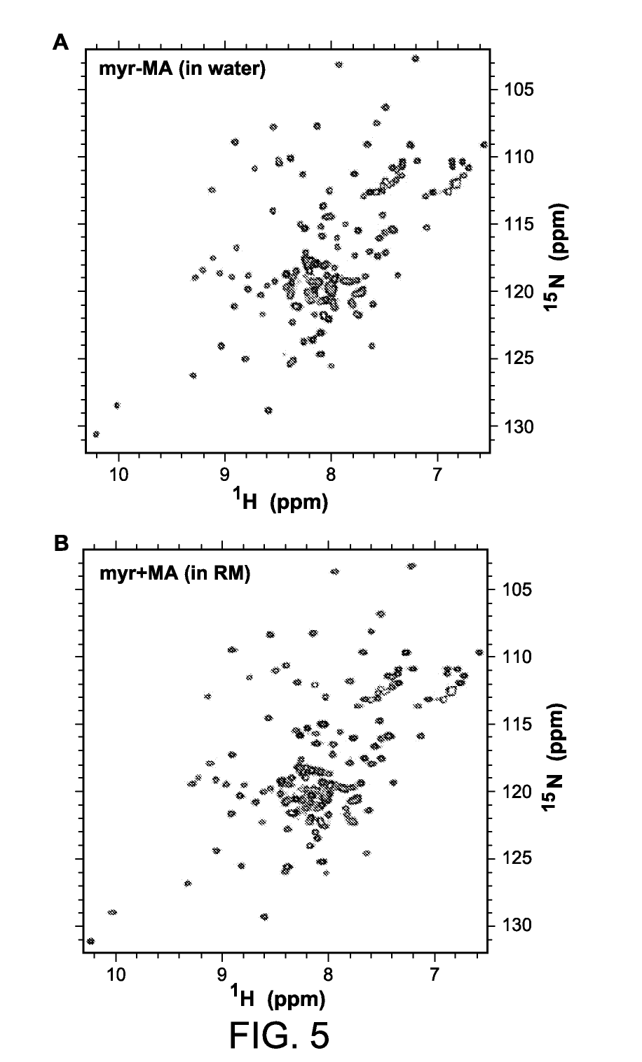

In the present study, the cetyl trimethyl ammonium bromide (CTAB) was used to provide a surfactant shell to mimic the membrane found in cells. This was facilitated by the fact that the surfactant tail length was close to half the membrane width in cell membranes, and also closely resembled the form of the lipid anchors. The Gag MA protein was combined with the chosen surfactant, and NMR analysis of the protein solubilized in the CTAB reverse micelle system was conducted. It was surprisingly discovered that the free solution ¹⁵N-HSQC spectrum of the myr-protein was essentially identical to that of the encapsulated myr+MA protein (FIG. 5). The only differences were at the attachment point of the myristoyl group. The most probable explanation is that the myristoyl group had been completely extruded from the protein and was embedded in the reverse micelle surfactant shell. FIG. 5 illustrates how the reverse micelle encapsulation of the myr+MA protein promotes and traps the extruded state (tantamount to a native membrane-activated state). FIG. 5A shows the ¹⁵N-HSQC spectrum of the MA protein without the myristoyl group, while FIG. 5B shows the ¹⁵N-HSQC spectrum of the myristoylated protein in the CTAB reverse micelle system. The spectra are virtually identical indicating that the myristoyl group is extruded and the protein has reorganized to the myr-structure. This latter state is impossible to visualize in free solution in such detail due to the poor solution properties of this state of the protein, i.e., extensive aggregation occurs.

Analysis of the NOESY confirmed that myr+MA protein had extruded the myristoyl group and adopted the myr-MA structure. The CTAB surfactant used to create the reverse micelle system is positively charged. Thus, the combined effect of the confined space of the reverse micelle (see Peterson, R. W., Anbalagan, K., Tommos, C., and Wand, A. J. (2004) Forced folding and structural analysis of metastable proteins. J Am Chem Soc 126, 9498-9) and the likely gain in free energy gained for burial of the myristoyl moiety in the CTAB shell (which is 16 carbons in length; see Peitzsch, R. M. and McLaughlin, S. (1993) Binding of acylated peptides and fatty acids to phospholipid vesicles: pertinence to myristoylated proteins. Biochemistry 32, 10436-43) was apparently sufficient to overcome any electrostatic repulsion between the positive surfactant headgroup and the positive patch commonly associated with matrix proteins of retroviruses. This represented a tremendously significant result as it indicates that the present invention provides a model system with which the myr+protein can be studied in the membrane activated state.

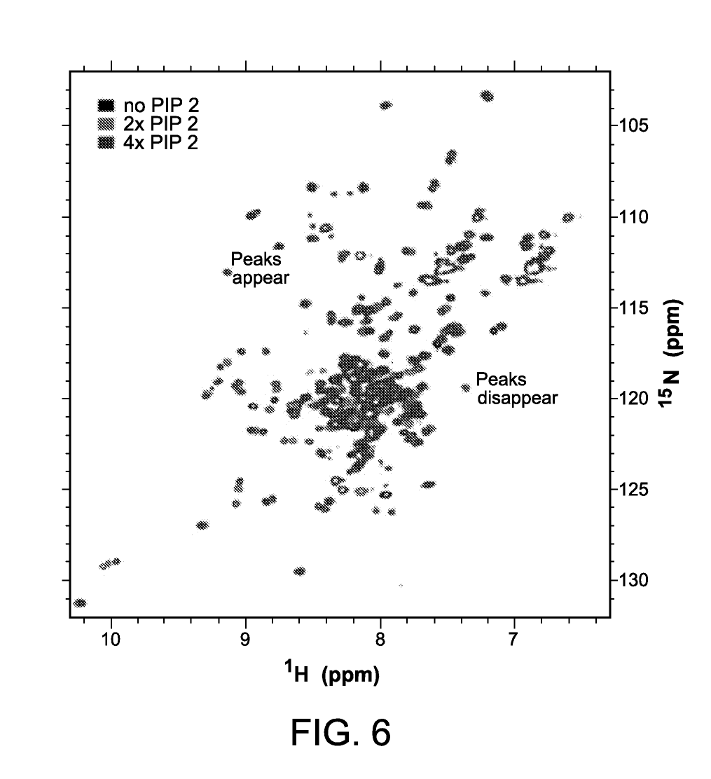

Native PI(4,5)P2, while not particularly soluble in solutions of empty (i.e., water-containing) reverse micelles, is rapidly solubilized by reverse micelles containing the myr+MA protein. This is a strong indication that the protein is picking up the added ligand. Indeed, the ¹⁵N-HSQC titration shows multiple cross peak shifts upon addition of PIP2, while some disappear and new crosspeaks appear (FIG. 6). The present techniques therefore appear to provide access to the physiologically relevant complex.

It is important to note that all spectra are of high quality. They indicate maintenance of relevant structure. Encapsulation/solubilization in accordance with the present invention serves as a means to provide a membrane mimic for burial of the myristoyl group and thereby avoids the debilitating properties of a solvent- (water-) exposed myristate chain. The instant inventions apply, inter alia, to research in structural biology. Specifically the study of membrane-anchoring proteins that have proven to be very difficult to study by other high resolution structural NMR due to the poorly behaved nature of this class of proteins. The use of the instant system permits detailed structural studies of this class of proteins. It also provides a high fidelity system with which to undertake binding assays, something that has heretofore proven difficult.

### Example 4

### Encapsulation of a Functional Membrane Associated Protein—Myristoylated Recoverin—into Reverse Micelles

Recoverin is the 23 kDa calcium binding protein that plays a crucial role in the visual phototransduction cascade. The protein is composed of four EF hand, Ca²⁺ binding motifs and a fatty acyl covalent attachment at the amino terminus Recoverin has been identified as the antigen in the auto-immune disease of the retina, cancer associated retinopathy (see Polans, A., et al. (1991) A photoreceptor calcium binding protein is recognized by autoantibodies obtained from patients with cancer-associated retinopathy. J. Cell Biol. 112, 981-989). The binding of Ca²⁺ to recoverin induces the attachment of myristoylated recoverin to the rod outer segment disk membranes. Solution NMR methods have been used to determine the structure of the Ca²⁺ free myristoylated recoverin (see Tanaka, T., et al. (1995) Sequestration of the membrane-targeting myristoyl group of recoverin in the calcium-free state. Nature 376, 444-447). The Ca²⁺ bound state proved to be a more difficult structural candidate. The cooperative binding of two Ca²⁺ ions causes the extrusion of the myristoyl group leading to the aggregation of the protein in the absence of a proper membrane attachment site. The solution structure of the Ca²⁺ bound state of recoverin was determined by using the 13-oxa analogue of the myristoyl group. The analogue allowed the recoverin to remain soluble in the absence of the membrane bilayer (see Ames, J., et al (1997) Molecular mechanics of calcium-myristoyl switches. Nature 389, 198-202). It would be useful to determine the structure of the myristoylated recoverin as it binds Ca²⁺ in the presence of a membrane mimic The association that occurs with the membrane could play a key role in the conformational switch as calcium is bound and recoverin is sequestered to the rod outer segment disk membranes. The instant reverse micelle technology offered the opportunity to probe the mechanics of the conformational switch in the sequential stages of calcium binding in the same membrane mimic environment.

The encapsulation of myristoylated recoverin into reverse micelles was achieved by preparing a concentrated (2.1 mM) solution of ¹⁵N labeled myristoylated recoverin in an aqueous buffer containing 25 mM NaCl, 10 mM tris-(hydroxymethyl)aminomethane (Tris), 3 mM threo-2,3-dihydroxy-1,4-dithiolbutane (DTT) at pH 7.0. The surfactant solution was premixed containing 100 mM hexadecyltrimethylammonium bromide (CTAB) solubilized in 6.5% vol/vol hexanol/d₁₂-pentane. The concentrated recoverin solution (20.5 μL) was next injected into the surfactant solution (750 μL) and vortexed vigourously until the solution remained clear. The resulting reverse micelle solution was 57 μM recoverin: 100 mM CTAB: 520 mM hexanol in pentane with a water loading (W₀) of 15. The myristoylated recoverin in reverse micelles could then be manipulated to form the calcium loaded conformation by adding 2 μL of a 40 mM CaCl₂ solution to the reverse micelle solution containing the Ca⁺² free form of myristoylated recoverin.

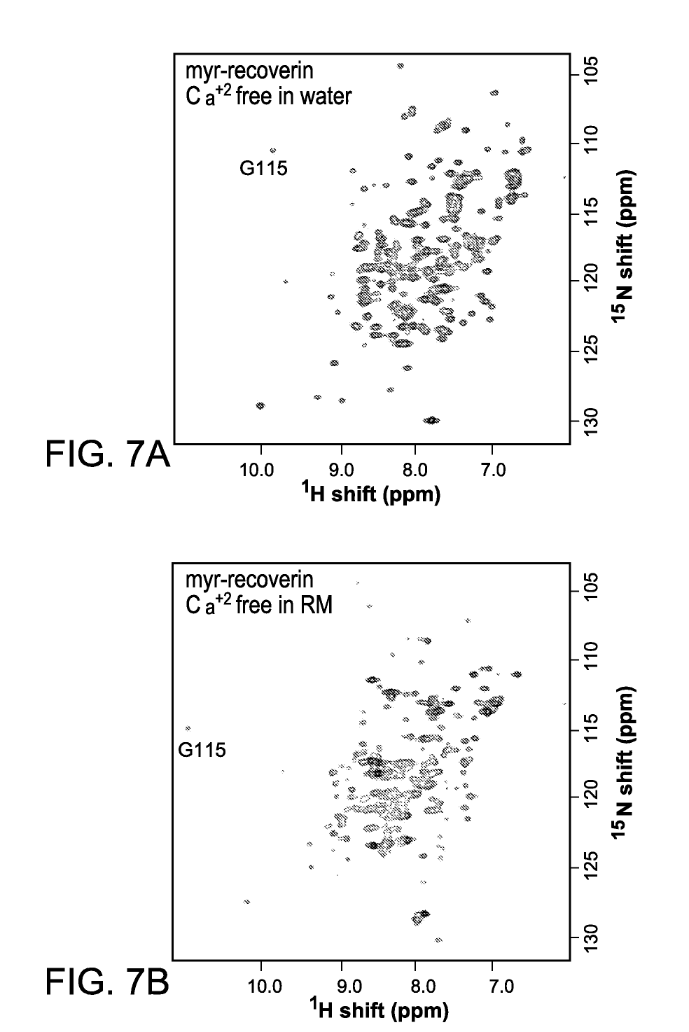

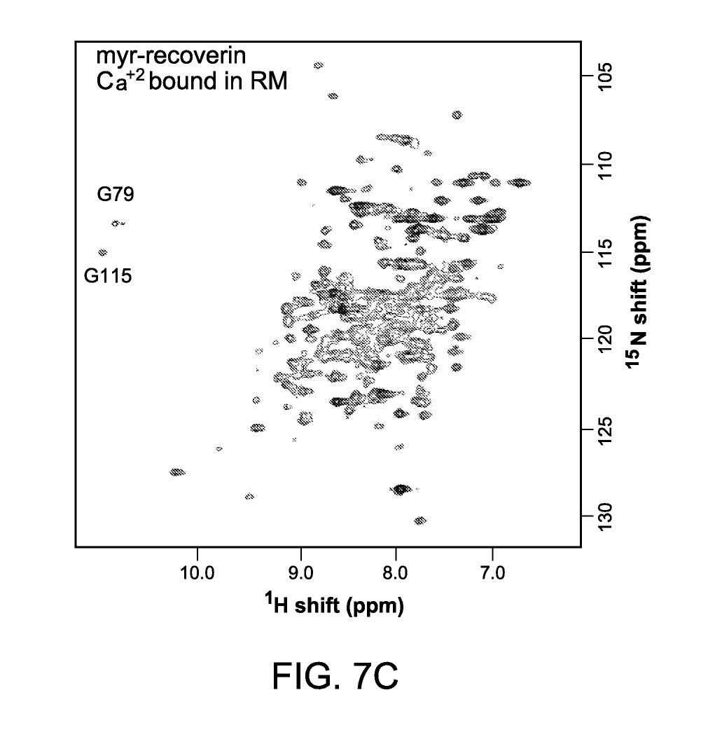

The resulting solutions were then subject to ¹⁵N HSQC spectral acquisitions. FIG. 7A shows the ¹⁵N-HSQC of the water solubilized Ca²⁺ free myristoylated recoverin. The beacon residues that are involved in the sequestration of the myristoyl chain are indicated on the spectra. The encapsulation of recoverin is shown in FIG. 7B. Similar to the HIV matrix protein, it appeared to be the case that the encapsulation process caused the extrusion of the myristoyl group into the lipid surfactant phase in the reverse micelle. The evidence was found in the extreme chemical shift change noted for the G115 residue. This same large down field shift was noted for the G115 residue in the Ca²⁺ bound state of the myristoyl analogue of recoverin in aqueous solution. The absence of G79 in both the water soluble ¹⁵N HSQC of Ca²⁺ free myristoylated recoverin (FIG. 7A) and the Ca²⁺ free reverse micelle spectrum of myristoylated recoverin (FIG. 7B) indicated that a similar conformational averaging is present at this site in both aqueous and reverse micelle environments. It was not until the addition of Ca²⁺ in the reverse micelle solution (FIG. 7C) that the resonance reappears in the same extreme shifted position determined for the 13-oxa myristoyl analogue (see Ames, J., et al. (2002) Structure and calcium-binding studies of recoverin mutant (E85Q) in an allosteric intermediate state. Biochemistry 41, 5776-5787.).

The disclosures of each patent, patent application and publication cited or described in this document are hereby incorporated herein by reference, in their entirety.

What is claimed:

1. A method for solubilizing a membrane protein comprising:
blending a first surfactant comprising an aqueous detergent with a sample of said membrane protein to form an aqueous detergent-protein mixture;
concentrating said mixture; and,
combining said concentrated mixture with an organic solvent and either a second surfactant or an additional quantity of said first surfactant to form a reverse micelle system in which said membrane protein is solubilized, and wherein said solubilized membrane protein is substantially homogeneous.

2. The method according to claim 1 wherein said membrane protein is an anchored membrane protein or an integral membrane protein.

3. The method according to claim 1 wherein said membrane protein is a recombinantly expressed protein.

4. The method according to claim 1 wherein the membrane protein is present in the reverse micelle system in an amount of at least about 0.1 mM.

5. The method according to claim 1 comprising concentrating said detergent-protein mixture by an amount necessary to render said mixture substantially dry.

6. The method according to claim 1 wherein said organic solvent comprises a low-viscosity alkane comprising fewer than 10 carbon atoms.

7. The method according to claim 1 wherein said first surfactant comprises at least one of lauryldimethylamine oxide, sodium bis(2-ethylhexyl) sulfosuccinate, cetyltrimethylammoniumbromide, and N,N-dimethyl-N,N-dihexadecyl ammonium bromide.

8. The method according to claim 1 comprising combining said concentrated mixture with a second surfactant, and wherein said second surfactant comprises an aqueous detergent or an alcohol.

9. The method according to claim 8 wherein said second surfactant comprises at least one of pentanol, hexanol, N,N-dimethyl-N,N-dihexadecyl ammonium bromide, or sodium bis(2-ethylhexyl) sulfosuccinate.

10. The method according to claim 1 comprising combining said concentrated mixture with a second surfactant, wherein said first surfactant comprises cetyltrimethylammoniumbromide and said second surfactant comprises N,N-dimethyl-N,N-dihexadecyl ammonium bromide, and wherein said first surfactant and said second surfactant are present in said reverse micelle system in about a 1:1 ratio.

11. A method for assessing a drug candidate comprising:
contacting said drug candidate with a membrane protein that is solubilized within a reverse micelle system in accordance with the method of claim 1; and
performing at least one of assessing the binding between said drug candidate and said membrane protein, determining whether said drug candidate modulates the conformation of said membrane protein, determining whether said drug candidate modulates the degradation characteristics of said membrane protein, and determining whether said drug candidate modulates post-translational modification of said membrane protein.

12. A substantially homogeneous membrane protein that is solubilized within a reverse micelle system comprising a first surfactant, the first surfactant comprising an aqueous detergent; an organic solvent; and, optionally, a second surfactant.

13. A method for performing spectroscopic analysis of a membrane protein comprising:
placing a substantially homogeneous sample of said membrane protein in a spectroscopic analytical instrument, wherein said membrane protein is solubilized within a reverse micelle system comprising a first surfactant, the first surfactant comprising an aqueous detergent; an organic solvent; and, optionally, a second surfactant;
and, performing spectroscopic analysis of said solubilized membrane protein.

14. The method according to claim 13 wherein said spectroscopic analysis comprises nuclear magnetic resonance analysis, and wherein the membrane protein is present in the reverse micelle system in an amount of at least about 0.1 mM.

15. A method for solubilizing an anchored membrane protein comprising combining said protein with a surfactant capable of forming reverse micelles.

16. The method according to claim 15 comprising combining said protein with said surfactant and an alcohol.

17. The method according to claim 15 wherein said surfactant comprises one or more of lauryldimethylamine oxide, sodium bis(2-ethylhexyl) sulfosuccinate, cetyltrimethylammoniumbromide, and N,N-dimethyl-N,N-dihexadecyl ammonium bromide.

18. The method according to claim 15 wherein said anchored membrane protein is a recombinantly expressed protein.

19. An anchored membrane protein that is solubilized within a reverse micelle system.

20. The solubilized anchored membrane protein according to claim 19 wherein said anchored membrane protein is a recombinantly expressed protein.

21. The solubilized anchored membrane protein according to claim 19 wherein said reverse micelle system comprises at least one of lauryldimethylamine oxide, sodium bis(2-ethylhexyl) sulfosuccinate, cetyltrimethylammoniumbromide, and N,N-dimethyl-N,N-dihexadecyl ammonium bromide.

22. A method for performing spectroscopic analysis of an anchored membrane protein comprising:
placing a sample of said anchored membrane protein in a spectroscopic analytical instrument, wherein said anchored membrane protein is solubilized within a reverse micelle system; and
performing spectroscopic analysis of said anchored membrane protein.

23. The method according to claim 22 wherein said spectroscopic analysis comprises nuclear magnetic resonance analysis, and wherein the anchored membrane protein is present in the reverse micelle system in an amount of at least about 0.1 mM.

24. A method for assessing a drug candidate comprising:
contacting said drug candidate with an anchored membrane protein that is solubilized within a reverse micelle system; and
performing at least one of assessing the binding between said drug candidate and said anchored membrane protein, determining whether said drug candidate modulates the conformation of said anchored membrane protein, determining whether said drug candidate modulates the degradation characteristics of said anchored membrane protein, and determining whether said drug candidate modulates post-translational modification of said anchored membrane protein.
